(19) United States
(12) Patent Application Publication
(10) Pub. No.: US 2004/0062779 A1
(43) Pub. Date: Apr. 1, 2004

(54) Dermatologic composition using ultra-fine/micronized 1-ascorbic acid and other antioxidant ingredients in a stabilized anhydrous vehicle

(76) Inventors: Jerry Whittemore, Los Angeles, CA (US); Robert Strom, Rancho Mirage, CA (US)

Correspondence Address: Jerry Whittemore, 3300 Shelby Dr., Los Angeles, CA, 90034, US

(21) Appl. No.: 10/256,022

(22) Filed: Sep. 26, 2002

(51) Int. Cl. A61K 31/375; A61K 31/74; A61K 9/00

(52) U.S. Cl. 424/400; 514/474

(57) ABSTRACT

This composition is a topical admixture of submicron vitamin C in percentages of 40.001%-70% by weight with exceptional biological activity, and exhibiting deep transdermal penetration.

Using sodium ascorbate admixed with the 1-ascorbic acid reduces the epidermal irritation of high concentrations of total vitamin C.

The sodium ascorbate naturally buffers the acidity of 100% 1-ascorbic acid Polyolprepolymer-2 enhances the epidermal penetration.

References Cited:
U.S. Patent Documents

| | | | |
|---|---|---|---|
| 2400171 | May, 1946 | Ruskin. | |
| 4209449 | June, 1980 | Mayhew et al. | |
| 4367157 | January, 1983 | Sherman. | |
| 4372874 | April, 1884 | Modrovich | |
| 4563346 | January, 1986 | Deckner. | |
| 4603046 | July, 1986 | Georgalas et al. | |
| 4784845 | November, 1988 | Desai et al | |
| 4847267 | July, 1989 | Deckner et al | |
| 5008100 | April, 1991 | Zecchino et al. | |
| 5141665 | August, 1992 | Sherman | |
| 5215976 | June, 1993 | Fost et al | |
| 5286719 | February, 1994 | Fost et al | |
| 5290481 | March, 1994 | Todd, Jr. | |
| 5296249 | March, 1994 | Todd, Jr. | |
| 5516506 | May, 1996 | Fogel | |
| 5587149 | December, 1996 | Punto et al. | |
| 566205 | September, 1997 | Ogawa et al | |
| 5663270 | September, 1997 | Richard et al. | |
| 5670160 | September, 1997 | Eggensperger. | |
| 5738859 | April, 1998 | Posner | |
| 5746945 | May, 1998 | Ryklin et al | |
| 5759523 | June, 1998 | Hughes et al. | |
| 5804168 | September, 1998 | Murad | |
| 5902591 | May, 1999 | Herstein | 424/401 |
| 6146664 | November, 2000 | Siddiqui | |
| 6361783 | May 2000 | Wolf, et al | |

Foreign Patent Documents

| | | |
|---|---|---|
| 0551030 | September, 1993 | SU. |
| WO 96/00060 | January, 1996 | WO. |

### OTHER REFERENCES:

Boehne, J W, “vitamins and other Nutrients,” Remington's Pharmaceutical Sciences, Mack Publishers, Easton, Pa. (1975) p. 987.

Darr et al., Topical Vitamin C Protects Porcine Skin From Ultraviolet Radiation-Induced Damage, British Journal of Dermatology 127:247-253 (1992).

VERIS Research Summary, The Role of Antioxidants in Skin Care and Protection, VERIS Research Information Service, (1997).

POLYOLPREPOLYMERS, Barnet brochure, Englewood Cliffs, N.J. (1999).

ACTIVE DELVERY SYSTEMS, Brooks Industries brochure, So. Plainfield, N.J. (2000).

SPECIALTY INGREDIENTS, Dragoco Brochure, Totowa, N.J.(2001).

AFR. brochure, Cognus, Ambler, Pa. (2002).

Mason, Joel B., Consequences of Altered Micronutrient Status, “Nutritional Diseases,” Cecil's Textbook of Medicine, Saunders, Philadelphia Pa. (1994) p. 1044.

Rieger, M. M., Oxidative Reactions in and on Skin: Mechanism and Prevention, Cosmetics and Toiletries 108:43-56 (1993).

Oxidative Stress, H. Sies, Ed., Academic Press, (1985).

Free Radicals, Aging and Degenertive Diseases, J. Johnson., R. Walford, D. Harmon and J. Miguel, Eds. Alan Liss, Inc., New York, (1986).

Biological Role of Reactive Oxygen Species In Skin, O. Hayaish, S. Inamujra and Y. Mayachi, Eds. (Elsevier Press, New York, (1987).

Idson, B., Vitamins and the Skin, Cosmetics and Toiletries 108:79-94 (1993).

DeRitter et al, Effect of Silica Gel on Stability and Biological Availability of Ascorbic Acid, Journal of Pharmaceutical Sciences, Phil, Pa. (1999).

Pinnell, S. Regulation of Collagen Biosynthesis by Ascorbic Acid: A Review (1999).

21 CFR part 333 (OTC Monograph-“Protectants-Final Rules.” (2000)

### DESCRIPTION

1. Field of the Invention

This invention concerns a topical formulation of submicron vitamin C particles that exhibits high biological activity, exhibits deep transdermal penetration, is less irritating than other vitamin C serums, and is totally stable to oxidation or hydrolysis from contamination by metallic copper ions, water and atmospheric oxygen.

2. Background of the Invention

For well over half a century pharmaceutical chemists have explored the factors that increase the stability of vitamin C in aqueous as well as anhydrous dosage forms. Many contaminants, including water (both hydronium and hydroxyl ions), simply open the lactone ring structure resulting in loss of activity and change to an unattractive brown color in the dosage form.

Ascorbate esters including ascorbyl palmitate, tetrahexadecyl ascorbate, magnesium ascorbyl phosphate, in addition to metallic ascorbate esters have been found to be stable in many dosage forms.

But these ascorbate species have two profound disadvantages in commercial use. (1) They are highly expensive, and (2) these large esters are not high in actual “vitamin C.” L-ascorbic acid and Sodium ascorbate are inexpensive and are “genuine vitamin C” in both layman's connotation and health professionals' denotation.

U.S. Pat. No. 2,400,171 (Ruskin) discloses the transition of the acid moiety of ascorbic acid to the zinc or calcium salt to effect stability of the vitamin C.

U.S. Pat. No. 4,367,157 discloses stabilizing vitamin C solutions using monothioglycerol.

U.S. Pat. No. 4,372,874 (Modrovich) discloses using a desiccant to an aqueous vitamin C preparation in order to isolate water from the lactone ring.

U.S. Pat. No. 5,140,043 (Darr et al.) discloses stabilizing about 1% vitamin C preparations with glycols with additional hydroxyalkylcellulose in an acid medium.

U.S. Pat. No. 5,296,249 (Todd) discloses micron particles of straight ascorbic acid in a medium where the straight acid was not soluble.

U.S. Pat. No. 5,308,621 (Taylor et al.) discloses particulate vitamin C in a transdermal product using glycols or petroleum jelly.

U.S. Pat. No 5,587,149 (Punto et al.) discloses a polyethylene-in-oil emulsion of ascorbic acid being used to protect the lactone ring.

U.S. Pat. No. 6,146,664 (Siddiqui) discloses a 0.1%40% straight ascorbic acid in a polyorganosiloxane gel vehicle.

U.S. Pat. No. 6,361,783 (Wolf, et al) discloses an emulsion system using vitamin c with both polyols and high molecular weight surfactant ethers.

Vitamin C offers significant value in ophthalmic and topical usage. Its penetration through the skin is well documented in the references. The ability of ascorbic acid to scavenge free radicals in a variety of age-related scenarios is well documented. In an article titled “Regulation of Collagen Biosynthesis by Ascorbic Acid: A Review” by S. Pinnell in Yale J. Biol. Med. 58:554 (1985), it was shown that ascorbic acid stimulates collagen synthesis in-vitro. This has enormous commercial value in the venerable fight against wrinkles in mature human skin.

Three facts about topical vitamin C are not controversial: (1) Vitamin C is valuable in treating skin conditions (particularly when rubbed into the layers of cells below the keratinocytes); (2) Vitamin C has very poor stability due to the opening of the lactone ring due to water contamination, oxygen contamination, and copper ion triggered catalysis; and then rubbed in.

### SUMMARY OF THE INVENTION

This invention is a composition whereby vitamin C species (ascorbic acid and/or sodium ascorbate) are micronized, using methods known to those familiar in the art of micronizing chemical granules into particles less than 25 m. mu. in mean diameter. These methods include jet milling and ball milling. Other methods of micronization are known to those skilled in the art of micronizing. Our invention includes the improvement of using 0.01-4% by weight of fumed silica. The most preferable percentage of fumed silica used is adding 0.3% by weight of the fumed silica during the first half (or first pass) of the micronization process and by adding an additional 0.3% by weight of fumed silica during the second half (or second pass) of the micronization process. The addition of fumed silica reduces particle agglomerization during the process. The use of fumed silica to prevent agglomerization in the micronization process adds the unexpected finding that fumed silica helps potentiate the physical stability (freedom from settling out of the vitamin C in the low viscosity silicone liquid base.

A key discovery of this invention is that using silicone species of low viscosity, preferably low viscosity dimethicone, low viscosity dimethiconol, low viscosity cyclopentasiloxane, low viscosity cetyl dimethicone, low viscosity phenyl trimethicone, low viscosity hexyl dimethicone (more preferably dimethicone 10-50 csk., most preferably dimethicone 20 csk); allows the creation of a cosmetically elegant, topical dermatological composition with 40.001%-70% by weight (most preferably 52-55% by weight) of true vitamin C species (1-ascorbic acid and sodium 1-ascorbate). Prior art using polyols and silicone gels as cosmetic bases produced unacceptably thick pastes with vitamin C concentrations far lower than our compositions.

The composition of our invention focuses on rigorous quality control of the low viscosity silicone fluid base to assure ultra low concentrations of water, oxygen and copper ions which trigger hydrolysis and breakage of the lactone ring of vitamin C.

One object of our invention was to use ultra high levels of vitamin C as an antioxidant to scavenge free radicals to fight wrinkles and the signs of aging. An additional object of our invention was to add additional and potentiating antioxidants to this admixture. Our invention includes, but not exclusively, the admixture of beta-glucon, beta carotene, zanthalene, soy flavonoids, soy isoflavonoids, thiotic acid (alpha lipoic acid), tocopherol acetate, tocopherol, lutein, green tea extract, and Paraguay green tea extract. Other similar additions are known to those informed in the art of free radical scavenging. The composition of our invention includes the use of polyolprepolymer-2 (preferably 0.01-10% by weight and most preferably 1.0-1.5% by weight) as an agent to potentiate the penetration of vitamin C micro particles into the human epidermis.

This invention offers these unexpected advantages:

1. Remarkably high percentages of particulate submicron vitamin C were obtained in a topical product. In the most preferred embodiment, the total vitamin C is 52%-55% by weight

2. Remarkable lack of skin irritation on human skin was observed because the 1-ascorbic acid is naturally buffered by the partial substitution of sodium ascorbate. The most preferred embodiment is a ratio of 1-ascorbic acid to sodium ascorbate of 2:1 to 1:1.

3. Surprisingly high transepidermal penetration of the finished preparation with the admixture of the polyolprepolyment-2 to the final preparation.

4. Surprising symbiosis was seen by adding about 1% by weight of fumed silica to the ascorbic acid-sodium ascorbic mixture during the jet milling or ball milling of the vitamin C granules into submicron size. This same fumed silica which helps the granules glide into the jet milling/ball milling process helps suspend the jet milled/ball-milled particles in the thin silicone base. This most preferred embodiment of 52% vitamin C submicron particles in fine dimethicone is stable for over three years

5. Prior arts including U.S. Pat. Nos. 6,146,664 (Siddiqui) and 5,587,149 (Punto et al.) focus on thick silicones and thick emulsions to gain long term stable suspensions. Our invention discloses the use of an elegant and thin dimethicone (preferred embodiment is dimethicone 26 csk or dimethicone 50 csk). There is absolutely no stickiness with ascorbic loadings up to 55% or 60% by weight. This embodiment is absolutely more cosmetically elegant and has more commercial value. And Siddiqui only discloses up to 40% by weight (with the irritation of total ascorbic acid-unbuffered with sodium ascorbate). Our discovery provide unexpectedly high ascorbate activity and the lack of irritation which is found with straight ascorbic acid. Our disclosure has a pH much closer to neutrality and is dramatically less irritating due to both the admixture of sodium ascorbate and the emollient activity of many of the antioxidants like zanthalene, category I protectant dimethicone and soy isoflavonoids.

6. Prior art U.S. Pat. No. 6,361,783 (Wolf, et al) focuses on an emulsion system combining common polyols with higher molecular weight surfactants to dissolve (most preferably 10.6% by weight of ascorbic acid). While this disclosure mentions high percentages of the ascorbate moiety, the “preferable” percentage of ascorbate is 10.6% by weight. Moreover, the use of high fatty alcohol-ceteareth 20 type compounds produce a thick, asphalt like composition that is not cosmetically acceptable and has less commercial value.

7. Surprising compatibility and synergism was found with this ascorbic-silicone suspension and most all currently fashionable anti-oxidants such as vitamin E, zanthalene, soy iso-flavonoids, green tea extract, lutein, alpha lipoic acid, Paraguay green tea, beta-glucon, beta-carotene, BHT, BHA and Propyl Gallate.

8. Polyolprepolymer-2 aids in the penetration of vitamin C species into the human epidermis.

What is claimed is:

1, A topical composition, comprising:
particulate vitamin C in the amount 40.0001%-70% by weight; and
an inert dimethicone topical serum, in the amount of 30-59.9999% by weight, wherein the silicone serum is in a sufficient amount to suspend the high amount of vitamin C, and the particulate vitamin C consists of chemically pure solids having a particle size of less than about 25 .mu.m.

2, The composition of claim 1, wherein the particulate vitamin C consists of essentially chemically pure 1-ascorbic acid.

3, The composition of claim 1, wherein the particulate vitamin C consists essentially of chemically pure sodium ascorbate.

4, The composition of claim 1, wherein the particulate vitamin C consists essentially of an admixture of 1-ascorbic acid and sodium ascorbate.

5, The composition of claim 1, wherein the particulate vitamin C consists of an admixture of 1-ascorbic acid, sodium ascorbate and fumed silica with the ratio of fumed silica to total vitamin C species being most preferably 0.14% by weight.

6, The composition of claim 1, wherein the dimethicone-serum is 100% dimethicone.

7, The composition of claim 1, wherein the dimethicone-serum is 100% dimethicone or dimethicone plus dimethiconol dissolved in cyclopentasiloxane.

8, The composition of claim 1, wherein the dimethicone-serum is replaced by 0.01-50% phenyl trimethicone, 0.01-50% cetyl dimethicone or 0.01-50% hexyl dimethicone.

9, The composition of claim 1, wherein the dimethicone serum is an admixture of dimethicone and dimethiconol.

10, The composition of claim 1, wherein the dimethicone-serum has the suspending agent fumed silica added in the amount of about 0.01-2% by weight, preferably 1% by weight.

11, The composition of claim 1, wherein the dimethicone serum is substantially free of copper ions which catalyzes oxidation of vitamin C species and breakdown of the lactone ring.

12, The composition of claim 1, wherein the dimethicone serum is substantially free of water and atmospheric oxygen, and in which the vitamin C species are totally insoluble.

13, The composition of claim 1, wherein about 1-2% by weight of the dimethicone-serum has been replaced by an admixture of anhydrous antioxidants including, but not limited to beta-glucon, beta-carotene, zanthalene, soy flavonoids, soy isoflavonoids, thiotic acid, tocopheryl acetate, tocopherol, lutein, green tea extract, Paraguay green tea extract.

14, The composition of claim 1, wherein the dimethicone base is admixed with polyolprepolymer-2 and, in which the percentage by weight of polyolprepolymer-2 in the low viscosity silicone base is 0.01% and 10%.
